(19) United States
(12) Patent Application Publication
(10) Pub. No.: US 2004/0241221 A1
(43) Pub. Date: Dec. 2, 2004

(54) Application method for enhanced oral assimilation through oral and gastrointestinal membranes, of liquids containing insulin, or other drugs, by enclosing them in stable, nanometer size, vesicles formed with a special combination of essential phospholipids (EPL) to minimize side effects and maximize absorption in oral applications

(76) Inventor: Nikolaus J. Smeh, Snowflake, AZ (US)

Correspondence Address: Mr. Nikolaus J. Smeh, 101 E 4th North, Snowflake, AZ, 85937, US

(21) Appl. No.: 10/449,228

(22) Filed: Jun. 2, 2003

(51) Int. Cl. A61K 38/28; A61K 9/127

(52) U.S. Cl. 424/450; 514/3

(57) ABSTRACT

The intend of this invention is to produce stable, non-leaking Essential Phospholipid (EPL) nano spheres for oral administration of insulin, chelating agents, and other substances, which are able to effectively bridge the gastrointestinal barrier for systemic treatment.

### BACKGROUND OF THE INVENTION

(1) Technical Field

Administration of Essential Phopholipid (EPL) Nano-encapsulated substances orally for the purpose of allowing transport through the oral and gastrointestinal membranes into the human or animal vascular system.

(2) Description of the Related Art

EPL Nano Capsules, also called vesicles or liposomes, have been used in the past to transport liquid substances through the epidermis through topical applications and also intravenously to deliver drugs. Applications to transport drugs orally and through the gastrointestinal tract have not been successful because the nano meter size capsules were prematurely destroyed in the Gastro-Instestinal tract and/or leaking encapsulated substances causing severe side effects to the stomach and the intestines. For example: about one half gram or more of Di-Sodium EDTA, given orally in an aqueous solution, cause immediate diarrhea while only less than 5% of the administered amount crosses into the blood stream. Even if encapsulated in specific phopholipids., like phoshatidylcholin, the same effect occurs and negligible amounts of f.i. Di-Sodium EDTA pass into the blood stream. The inventor has developed a specific combination different kinds of natural phospholipids which allow close to 90% of the administered solution to pass into the vascular system, without side effects in the gastrointestinal tract.

### BRIEF SUMMARY OF THE INVENTION

This invention relates to a novel method of creating stable Essential Phospholipid nano-spheres for enclosing insulin, or other substances. These micro-spheres are generated with the aid of special combinations of Essential Phospholipids. The property of these special combinations are such that the EPL micro-spheres become more stable in the gastrointestinal tract and avoid leakage of the encapsulated substance and the associated side effects.

### BRIEF DESCRIPTION OF THE SEVERAL VIEWS OF THE DRAWINGS

N/A

### DETAILED DESCRIPTION OF THE INVENTION

This invention relates to a novel method of creating stable Essential Phospholipid nano-spheres for encapsulating insulin and other drugs. These nano-spheres are generated with the aid of special combinations of Essential Phospholipids. The property of these special combinations are such that the EPL nano-spheres become more stable in the gastrointestinal tract and avoid leakage of the encapsulated substance. Since many of the encapsulated substances may cause serious side effects in the gastrointestinal tract when taken in large doses, like insulin or Di-Sodium EDTA, and vitamin C, stability of the micro-spheres is of great importance.

As a typical example we will describe the effect of Di-Sodium EDTA on the human system and especially on the gastrointestinal tract. Di-Sodium EDTA is extensively used in detoxification of the human system, especially for the removal of heavy metals. Di-Sodium EDTA is a very effective chelator, i.e. it captures dangerous heavy metal ions and allows the body to discharge them harmlessly through the kidneys. Up to now this is accomplished by dripping solutions containing 1-3 gram of Di-Sodium EDTA per treatment into the veins of the patient requiring 30 and more treatments for a single detoxifications.

When as little as 0.5 gram dose of Di-Sodium EDTA is taken orally with water, the bowels are emptied promptly and diarrhea results. Only less than 5% of the ingested dose makes it into the blood stream. When 2 gram or more of Di-Sodium EDTA is nano encapsulated using a single EPL, such as Phosphotidylcholin (the most abundant EPL), the same gastro-intestinal effect of diarrhea is observed, a clear indication that the microspheres consisting of only Phosphotidylcholine are leaking and are destroyed early in the digestive process.

However, when a special combination of EPLs is used, as developed by the inventor, to form nano-spheres, over 90% of the Di-Sodium EDTA is found in the bloodstream and no side effects on the gastrointestinal system are observed. This is also the case with other substances taken in large doses, such as vitamin C (10 gram per dose). To substantiate this claim, the inventor caused a clinical test to be performed to verify the passage of Di-Sodium EDTA from the gastrointestinal tract into the blood stream using special combinations of EPLs. This Clinical Test is unpublished as of this date. The results are as follows:

Clinical Absorption Study of Orally Administered Disodium EDTA on 16 Patients Performed by Dr. Daniel Dugi.

The purpose of this study is to prove the safety and effectiveness of using di-sodium EDTA orally in an essential phospholipid (EPL) microsphere delivery system, effectively bridging the intestinal barrier, for systemic heavy metal detoxification. This novel new oral form of di-sodium EDTA was developed by Nikolaus J. Smeh and tested clinically with sixteen patients. A baseline 24 hour urine was initially collected followed by provocation of two fluid ounces of a Lipoflow EDTA concentrate in fruit juice, containing one gram of di-sodium EDTA micro encapsulated in essential phospholipids. Two successive twenty-four hour urine collections followed the initial provocation. Excellent maximum increases in urine excretion over baseline control results were obtained: Aluminum >>100%, 960 ug/24 hr, Antimony 50%, Arsenic 1,558%, Bismuth 6,801%, Cadmium 233%, Lead 4,333%, Mercury 312%, Nickel 1,988%, Platinum >100%: 0.05 ug/24 hours, Thallium 300%, Thorium >100%: 0.05 ug/24 hours, Tin 1,980%, Tungsten 150% and Uranium >100%: 0.09 ug/24 hours.

1. Any natural or synthetic oral or topical delivery system using a special combination of Essential Phospholipids to nano encapsulate Insulin and other drugs. The new combination used to form stable microspheres is obtained by combining the following Phospholipids in various proportions:
(1) Phosphatidylcholine (PC)
(2) Phosphatidylethanolamine (PE)
(3) Phosphatidylinositol (PI)
(4) Minor Phospholipids

2. Any natural or synthetic oral delivery system using a special combination of Essential Phospholipids, as defined in claim 1, to nano encapsulate vitamin C from any source intended for enhanced oral assimilation for the purpose of passing these substances into the vascular system through the gastrointestinal tract.

3. Any natural or synthetic oral delivery system using a special combination of Essential Phospholipids, as defined in claim 1, to micro encapsulate ethylenediaminetetraacctic acid chelates intended for enhanced oral assimilation for the purpose of passing these substances into the vascular system through the gastrointestinal tract.
